(19) United States
(12) Patent Application Publication
(10) Pub. No.: US 2025/0205156 A1
(43) Pub. Date: Jun. 26, 2025

(54) FORMULATION FOR TRANSDERMAL DELIVERY

(71) Applicants: Dow Global Technologies LLC, Midland, MI (US); Rohm and Haas Company, Collegeville, PA (US)

(72) Inventors: Ashish Jayaraman, Conchohocken, PA (US); Daniel S. Miller, Phoenixville, PA (US); Xue Chen, Manvel, TX (US)

(21) Appl. No.: 18/847,302

(22) PCT Filed: May 30, 2023
(86) PCT No.: PCT/US2023/067592

§ 371 Date: Sep. 16, 2024

Related U.S. Application Data
(60) Provisional application No. 63/348,529, filed on Jun. 3, 2022.

(51) Int. Cl. A61K 9/107; A61K 9/00; A61K 47/10; A61K 47/14; A61K 47/44

(52) U.S. Cl. CPC A61K 9/107; A61K 9/0014; A61K 47/10; A61K 47/14; A61K 47/44

(57) ABSTRACT

Stable microemulsions that can contain a broad range of water content can be made using an organic mixture that contains: a) from 5 to 60 weight percent of a fatty alcohol or fatty ester; b) from 14 to 40 weight percent of ethoxylated castor oil; and c) from 20 to 65 weight percent of a cosolvent selected from the group consisting of lower alkyl diethylene glycol ether, lower alkyl triethylene glycol ether and propylene glycol diacetate; and d) from 5 to 25 weight percent of a chemical penetration enhancer for skin.

### FIELD

This invention relates to formulations for transdermal administration of pharmaceuticals.

### INTRODUCTION

It is known to administer pharmaceutical drugs by dissolving them in a transdermal formulation that is applied to the skin. The formulation is absorbed through the skin by osmosis and carries the dissolved drugs with it.

Many common formulations for transdermal delivery are microemulsions that comprise:
1. An aqueous solvent that is suitable for topical application and transdermal delivery;
2. An organic solvent that is suitable for topical application and transdermal delivery;
3. A surfactant or surfactant mixture;
4. One or more chemical penetration-enhancing additives; and
5. The drug to be delivered.

The microemulsion contains micelles in the dispersed phase that have an average size of no more than about 300 nm. In some cases the microemulsion is an “oil-in-water” microemulsion, in which the dispersed phase is organic and the continuous phase is aqueous. In some cases the microemulsion is a “water-in-oil” microemulsion, in which the dispersed phase is aqueous and the continuous phase is organic. Microemulsions used for transdermal delivery are described in the article: Lopes, Overcoming the Cutaneous Barrier with Microemulsions, 6 Pharmaceutics 52-77 (2014)

Microemulsions used for transdermal delivery must meet many competing requirements to be effective. The microemulsion must dissolve the necessary drug in a concentration high enough to be pharmaceutically effective. The microemulsion must absorb through the skin in high enough quantities to be pharmaceutically effective. The microemulsion must remain on the skin without evaporating during the time that the drug is expected to be absorbed, which may be a few hours for a slow-release formulation. The microemulsion must be shelf-stable long enough to manufactured, transported, stored and used without separating into oil and water phases. The microemulsion must contain only ingredients that are approved (or can readily be approved) by regulatory authorities like the FDA for human contact.

Many microemulsions have difficulty meeting all these requirements. Different drugs and different chemical penetration-enhancing additives require different ratios of organic and aqueous solvent in the microemulsion to achieve their best solubility and skin-penetration. However, the need for slow evaporation limits or excludes the use of light organic components that blend well with water. As a result, many emulsifier packages form stable microemulsions only in a narrow range of organic to aqueous ratios. The best organic-to-water ratio for transdermal delivery of a particular drug may lie outside the ratio that a specific organic component can achieve.

What is needed is an organic composition that can maintain a stable microemulsion across a broad range of organic to aqueous ratios, so that the organic composition can be formulated to the best advantage for a wide variety of drugs.

### SUMMARY

We have discovered that an organic composition that contains fatty alcohol or ester, ethoxylated castor oil and specific cosolvents can maintain a stable water-in-oil microemulsion across a relatively wide range of water-to-organic ratios.

One aspect of the present invention is an organic composition that comprises:
a) from 5 to 60 weight percent of fatty alcohol or fatty ester;
b) from 14 to 40 weight percent of ethoxylated castor oil; and
c) from 20 to 65 weight percent of a cosolvent selected from the group consisting of lower alkyl diethylene glycol ether, lower alkyl triethylene glycol ether and propylene glycol diacetate; and
d) from 5 to 25 weight percent of a chemical penetration enhancer for skin, wherein weight percentages are based on the weight of the organic composition.

A second aspect of the present invention is a microemulsion that contains from 1 to 40 weight percent water and from 60 to 99 weight percent of an organic composition as previously described.

A third aspect of the present invention is a method to administer a drug by applying to skin a microemulsion as previously described, which contains a dissolved drug in a pharmaceutically effective concentration.

The organic composition of this invention can form stable microemulsions with a broad range of water contents, such as from 1 to 30 weight percent, using only materials that are approved for medical use.

### BRIEF DESCRIPTION OF THE DRAWINGS

Drawings 1-13 each contain a three-phase diagram illustrating formulations that contain 1 to 10 weight parts in each diagram of (a) water, (b) isopropyl palmitate (IPP, a fatty ester), and (c) a mixture of ethoxylated castor oil, cosolvent and glyceryl oleate (a non-ionic surfactant skin permeation enhancer) (SCoS). Drawings 1-6 illustrate examples of the invention, and Drawings 7-13 illustrate comparative examples. In each diagram, “x” indicates a formulation that forms a stable microemulsion, and “o” indicates a formulation that does not form a stable microemulsion, under the conditions of stated in the Examples.

A detailed description of each formulation is contained in the working examples.

### DETAILED DESCRIPTION

### Organic Compositions

Organic compositions of this invention contain at least one fatty alcohol or ester, ethoxylated castor oil, at least one specific cosolvent described previously and a chemical penetration enhancer for skin. The organic components should be selected to be suitable for a transdermal drug delivery system. In some embodiments, it will be advantageous for all components of the organic system to be FDA approved for transdermal delivery; in other embodiments, one or more components may be selected that are capable of being approved later for transdermal delivery.

One component in the organic composition is fatty alcohol or a fatty ester. In some embodiments, the fatty component of the fatty alcohol or fatty ester comprises on average at least 4 carbon atoms or at least 8 carbon atoms or at least 10 carbon atoms or at least 12 carbon atoms. In some embodiments, the fatty component comprises on average at most 32 carbon atoms or at most 24 carbon atoms or at most 18 carbon atoms or at most 16 carbon atoms or at most 12 carbon atoms. Examples of suitable fatty alcohols include lauryl alcohol, myristyl alcohol, cetyl alcohol, stearyl alcohol, heptadecyl alcohol, pentadecyl alcohol and mixtures of these alcohols. In some embodiments, the fatty ester is an ester of a suitable fatty alcohol as previously described and lower carboxylic acid having up to 6 carbon atoms, such as acetic acid, propionic acid or butyric acid. In some embodiments, the fatty ester is a lower alkyl ester of a fatty acid. Examples of the fatty acid component include palmitic acid, myristic acid, lauric acid, coconut acid, behenic acid, stearic acid, oleic acid, undecylenic acid, caprylic acid and soyabean oil and mixtures of these. In some embodiments, the lower alkyl component linked to the fatty acid contains up to 8 carbon atoms or 6 carbon atoms or up to 4 carbon atoms; examples include methyl, ethyl, n-propyl, iso-propyl, n-butyl, isobutyl and t-butyl groups. Exemplary esters for use in the organic solvent include isopropyl palmitate, isopropyl myristate, butyl palmitate and butyl myristate, glyceryl stearate, ascorbyl palmitate, cetyl palmitate, ethyl myristate, glyceryl palmitate, and sorbitan monopalmitate and mixtures of these.

Suitable fatty alcohols and fatty esters are known and commercially available, such as from Aldrich Sigma, Kraft Chemical and Alfa Chemistry. The fatty alcohol or ester can comprise a single fatty alcohol or ester. Alternately, it can compromise blends that contain two or more fatty alcohols and/or esters.

One component of the organic composition is an ethoxylated castor oil. Ethoxylated castor oils are known and commercially-available, such as under the trademarks TERGITOL™ and Kolliphor. Ethoxylated castor oils are the product of a reaction of ethylene oxide with castor oil. Castor oil is a triglyceride, an ester derived from glycerol and three fatty acids. In castor oil, the predominant fatty acid is ricinoleic acid, which has a hydroxyl group pendant from the aliphatic chain. The hydroxyl group can initiate polymerization of ethylene oxide in the presence of a catalyst such as a base, resulting in a poly(ethylene glycol) chain grafted to the castor oil at the site of hydroxyl group. The polyethylene glycol chain is characterized by repeating ethylene glycol units having the formula in Formula 1:

—(—O—C₂H₄—)ₓ—OH (1)
wherein x is a number of repeating units. In some embodiments, the average degree of polymerization (number of repeating units “x”) in the poly(ethylene glycol) chains grafted to the castor oil is at least 20 or at least 25 or at least 27. In some embodiments, the average degree of polymerization in the poly(ethylene glycol) chains grafted to the castor oil is at most 50 or at most 42 or at most 40 or at most 38. In some embodiments, the ethoxylated castor oil contains saturated polymer chains, and in some embodiments the ethoxylated castor oil contains some unsaturation.

One component of the organic composition is at least one cosolvent selected from the group consisting of lower alkyl diethylene glycol ether, lower alkyl triethylene glycol ether and propylene glycol diacetate Examples of suitable cosolvents correspond to one of Formula 2(a) or 2(b)

R¹—(—O—C₂R²₄—)ₙ—OH 2(a)

C₂H₅—CO—O—C₃H₆—O—CO—C₂H₅ 2(b)
wherein R¹ is an aliphatic group comprising from 1-4 carbon atoms, each R² is independently hydrogen or an alkyl group comprising from 1 to 4 carbon atoms, and n is a number of repeating units that is on average greater than 1 and less than 4.

In some embodiments R¹ is alkyl. In some embodiments R¹ is an ethyl group, and in some embodiments R¹ is an n-propyl group or an isopropyl group.

In some embodiments, each R² is selected such that, on average, the aggregate number of carbon atoms in the R² groups attached to each repeating alkyl ether unit is from 0 to 8 or from 0 to 6 or from 0 to 4 or from 0 to 2 or from 0 to 1. In some embodiments, at least 90 percent of the repeating alkoxy units are ethoxy units.

In some embodiments, the number of repeating alkyl ether units (n) is at least 1.5 or at least 1.75 or at least 2. In some embodiments, the number of repeating alkyl ether units is at most 3.5 or at most 3.25 or at most 3.

Examples of suitable cosolvents include lower alkyl diethylene glycol ethers, such as diethylene glycol monoethyl ether, lower alkyl triethylene glycol ethers such as triethylene glycol monoethyl ether, and propylene glycol diacetate.

Diethylene glycol monoethyl ether [C₂H₅(OCH₂CH₂)₂OH] is commercially available under the trademarks CARBITOL™ and Transcutol; it is FDA approved for skin-contact. Propylene glycol diacetate is commercially available under the trademark DOWANOL™ PGDA. Other alkoxylated glycol ethers can be produced by the reaction of ethylene oxide with an alcohol in the presence of a catalyst such as a base. In some embodiments, the alcohol is an aliphatic lower alcohol having from 1-4 carbon atoms, such as methanol, ethanol, n-propanol, isopropanol, n butanol, isobutanol or t-butanol. In some embodiments the alcohol is saturated. The alcohol corresponds to the formula: R¹—OH wherein R¹ is an aliphatic group comprising from 1-4 carbon atoms.

One component of the organic composition is a chemical penetration enhancer for skin (CPE). Chemical penetration enhancers are known and commercially available. They are described in publications such as Vasyuchenko et al., CPE-DB: An Open Database of Chemical Penetration Enhancers, 2021 Pharmaceutics 13, 66. https://doi.org/10.3390/pharmaceutics13010066; Mathur, et al.: Physical and Chemical Penetration Enhancers in Transdermal Drug Delivery System, Asian Journal of Pharmaceutics—July-September 2010 at 173-183; Songkro, An Overview of Skin Penetration Enhancers, 31 (3) Songklanakarin J. Sci. Technol. at 299-321 (May-June 2009); and Som et al., Status of Surfactants as Penetration Enhancers in Transdermal Drug Delivery, 4(1) Journal of Pharmacy and Bioallied Sciences at 2-9 (January-March 2012)

Some embodiments of CPE fall within the broad categories of:
Solvents such as ethanol, propylene glycol, diethylene glycol monoethyl ether, oleic acid and dimethylsulfoxide,
Terpenes such as menthol, nerol, camphor and methyl salicylate, and
Surfactants such as Polysorbate 80, sodium dodecyl sulfate, sucrose monolaurate, glyceryl oleate, benzalkonium chloride, polyoxyl 40 hydrogenated castor oil, didecyldimethylammonium bromide (DDAB) and didecyltrimethylammonium bromide (DTAB).

In some embodiments the CPE is a surfactant. In some embodiments, the CPE is a non-ionic surfactant. Examples of suitable non-ionic surfactants include alkoxylated fatty alcohols such as ceteth-20 and laureth-23, alkoxylated fatty acid esters such as PEG-100 stearate, block copolymers of ethylene oxide and propylene oxide such as Poloxamer 124, sucrose monolaurate, glyceryl oleate, 1-dodecylazacycloheptan-2-one, 2-n-nonyl-1,3-dioxolane and fatty amines. CPEs are commercially available, such as under the following trademarks: Pationic, Azone and SEPA.

Organic compositions of the present invention contain from 5 to 60 weight percent of the fatty alcohol or fatty ester. In some embodiments, the organic composition contains no more than 50 weight percent of the fatty alcohol or fatty ester or no more than 40 weight percent or no more than 36 weight percent or no more than 30 weight percent or no more than 25 weight percent or no more than 20 weight percent. In some embodiments, the organic composition contains at least 7 weight percent of the fatty alcohol or fatty ester or at least 8 weight percent or at least 9 weight percent.

Organic compositions of the present invention contain from 14 to 40 weight percent of ethoxylated castor oil. In some embodiments, the organic composition contains no more than 38 weight percent of ethoxylated castor oil or no more than 35 weight percent or no more than 30 weight percent or no more than 25 weight percent. In some embodiments, the organic composition contains at least 16 weight percent of ethoxylated castor oil or at least 18 weight percent or at least 20 weight percent.

Organic compositions of the present invention contain from 20 to 65 weight percent of a cosolvent selected from the group consisting of lower alkyl diethylene glycol ether, lower alkyl triethylene glycol ether and propylene glycol diacetate. In some embodiments, the organic composition contains no more than 62 weight percent of the cosolvent or no more than 60 weight percent or no more than 55 weight percent. In some embodiments, the organic composition contains at least 22 weight percent of the cosolvent or at least 25 weight percent or at least 30 weight percent.

Organic compositions of the present invention contain from 5 to 25 weight percent of a chemical penetration enhancer for skin (CPE). In some embodiments, the organic composition contains no more than 20 weight percent of the CPE or no more than 18 weight percent or no more than 15 weight percent or no more than 12 weight percent. In some embodiments, the organic composition contains at least 7 weight percent of the CPE or at least 9 weight percent or at least 10 weight percent.

In some embodiments the weight ratio of cosolvent to ethoxylated castor oil is at least 2:10 or at least 4:10 or at least 5:10 or at least 55:100. In some embodiments the weight ratio of cosolvent to ethoxylated castor oil is at most 10:10 or at most 8:10 or at most 7:10 or at most 65:100.

In some embodiments, the ratio of ethoxylated castor oil to CPE is at least 1:1 or at least 15:10 or at least 18:10. In some embodiments, the ratio of ethoxylated castor oil to CPE is at most 5:1 or at most 3:1 or at most 18:10.

In some examples, the organic composition contains from 0.5 to 0.8 parts cosolvent per 1 part ethoxylated castor oil and CPE, by weight, and the components are in the following ratios:
At least 6 weight percent fatty alcohol or ester or at least 7 weight percent or at least 8 weight percent. At most 40 weight percent fatty alcohol or ester or at most 30 weight percent or at most 20 weight percent or at most 15 weight percent or at most 10 weight percent.
At least 25 weight ethoxylated castor oil or at least 32 weight percent or at least 35 weight percent. At most 45 weight percent ethoxylated castor oil or at most 40 weight percent.
At least 20 weight percent cosolvent or at least 24 weight percent or at least 30 weight percent. At most 40 weight percent cosolvent or at most 35 weight percent.
At least 10 weight percent CPE or at least 13 weight percent or at least 15 weight percent or at least 18 weight percent. At most 22 weight percent CPE or at most 20 weight percent.

In some examples, the organic composition contains from 0.8 to 1.2 parts cosolvent per 1 part ethoxylated castor oil and CPE, by weight, and the components are in the following ratios:
At least 6 weight percent fatty alcohol or ester or at least 7 weight percent or at least 8 weight percent. At most 40 weight percent fatty alcohol or ester or at most 30 weight percent or at most 20 weight percent or at most 15 weight percent or at most 10 weight percent.
At least 20 weight ethoxylated castor oil or at least 25 weight percent or at least 28 weight percent. At most 35 weight percent ethoxylated castor oil or at most 32 weight percent.
At least 30 weight percent cosolvent or at least 35 weight percent or at least 40 weight percent. At most 50 weight percent cosolvent or at most 45 weight percent.
At least 10 weight percent CPE or at least 11 weight percent or at least 12 weight percent. At most 20 weight percent CPE or at most 18 weight percent.

In some examples, the organic composition contains from 1.2 to 1.75 parts cosolvent per 1 part ethoxylated castor oil and CPE, by weight, and the components are in the following ratios:
At least 6 weight percent fatty alcohol or ester or at least 7 weight percent or at least 8 weight percent. At most 30 weight percent fatty alcohol or ester or at most 27 weight percent or at most 25 weight percent.
At least 18 weight ethoxylated castor oil or at least 20 weight percent or at least 21 weight percent. At most 30 weight percent ethoxylated castor oil or at most 25 weight percent.
At least 40 weight percent cosolvent or at least 42 weight percent or at least 45 weight percent or at least 48 percent. At most 60 weight percent cosolvent or at most 58 weight percent.
At least 8 weight percent CPE or at least 9 weight percent or at least 10 weight percent. At most 15 weight percent CPE or at most 13 weight percent.

In some examples, the organic composition contains from 1.75 to 2.5 parts cosolvent per 1 part ethoxylated castor oil and CPE, by weight, and the components are in the following ratios:
At least 6 weight percent fatty alcohol or ester or at least 7 weight percent or at least 8 weight percent. At most 40 weight percent fatty alcohol or ester or at most 30 weight percent or at most 20 weight percent or at most 15 weight percent or at most 10 weight percent.
At least 15 weight ethoxylated castor oil or at least 17 weight percent or at least 18 weight percent. At most 25 weight percent ethoxylated castor oil or at most 22 weight percent.
At least 45 weight percent cosolvent or at least 55 weight percent or at least 59 weight percent. At most 65 weight percent cosolvent or at most 62 weight percent.
At least 5 weight percent CPE or at least 6 weight percent or at least 8 weight percent. At most 15 weight percent CPE or at most 12 weight percent.

In some embodiments, the organic composition is capable of forming a stable water-in-oil microemulsion over a range of from 5 to 10 weight percent water or 5 to 15 weight percent water or 5 to 20 weight percent water or 5 to 25 weight percent water or 5 to 30 weight percent water or 5 to 35 weight percent water. In some embodiments, the organic composition is capable of forming a stable water-in-oil microemulsion over a range of from 10 to 20 weight percent water or 10 to 25 weight percent water or 10 to 30 weight percent water. “Stable microemulsion” means that the microemulsion can be stored at about 25° C. for at least one week without substantial destabilization. “Substantial destabilization” means that the composition shows observable signs of phase separation or creaming to two or more liquid layers (different layers of emulsion, each of which is rich in either the oil or the water phase).

In some examples, the organic composition can form a stable microemulsion with water in water concentrations of from 5 to 30 weight percent, and the components are in the following ratios:
At least 6 weight percent fatty alcohol or ester or at least 7 weight percent or at least 8 weight percent. At most 25 weight percent fatty alcohol or ester or at most 20 weight percent or at most 20 weight percent.
At least 20 weight ethoxylated castor oil or at least 22 weight percent. At most 30 weight percent ethoxylated castor oil or at most 25 weight percent.
At least 45 weight percent cosolvent or at least 50 weight percent. At most 65 weight percent cosolvent or at most 62 weight percent.
At least 8 weight percent CPE or at least 9 weight percent. At most 15 weight percent CPE or at most 13 weight percent.

In some examples, the organic composition can form a stable microemulsion with water in water concentrations of from 5 to 25 weight percent, and the components are in the following ratios:
At least 6 weight percent fatty alcohol or ester or at least 7 weight percent or at least 8 weight percent. At most 25 weight percent fatty alcohol or ester or at most 20 weight percent or at most 20 weight percent.
At least 25 weight ethoxylated castor oil or at least 27 weight percent or at least 30 weight percent. At most 40 weight percent ethoxylated castor oil or at most 38 weight percent.
At least 28 weight percent cosolvent or at least 30 weight percent or at least 31 weight percent. At most 50 weight percent cosolvent or at most 45 weight percent.
At least 10 weight percent CPE or at least 11 weight percent. At most 18 weight percent CPE or at most 15 weight percent.

In some embodiments, it is advantageous for the organic components to be selected so that the organic composition is liquid and non-volatile at skin-temperature. This permits the composition and its water-in-oil microemulsions to be readily applied to skin and to remain on the skin in liquid form for a time suitable to deliver a drug, such as at least 1 hour or at least 2 hours or at least 3 hours or at least 4 hours. In some embodiments, the organic composition melts at a temperature below 30° C. or below 25° C. or below 20° C. or below 10° C. or below 0° C. In some embodiments, the organic composition boils at a temperature above 50° C. or above 75° C. or above 100° C. or above 125° C. or above 150° C.

### Emulsions

The organic compositions previously described are made into microemulsions by mechanically mixing with water until a microemulsion forms. In some embodiments, the mixing may be enhanced by sonication. In some embodiments, the temperature of blending does not exceed 90° C. or 60° C. or 40° C.

In some embodiments, the pH of the water is at least 4 or at least 4.5 or at 4.75 or at least 5. In some embodiments, the pH of the aqueous solvent is at most 8 or at most 7 or at most 6.5.

In some embodiments, the microemulsion is an oil-in-water microemulsion. Some embodiments of oil-in-water microemulsion may contain at least 50 weight percent water and at least 20 weight percent organic composition.

In some embodiments, the microemulsion is a water-in-oil microemulsion. Some examples of water-in oil microemulsion contain from 1 to 40 weight percent water and from 60 to 99 weight percent of the organic composition. In some embodiments the microemulsion contains at least 2 weight percent water or at least 5 weight percent or at least 10 weight percent or at least 15 weight percent or at least 20 weight percent. In some embodiments the microemulsion contains at most 36 weight percent water or at most 32 weight percent or at most 30 weight percent or at most 25 weight percent.

In some embodiments of the microemulsion, the dispersed aqueous phase has average particle size of at most 300 nm. In some embodiments, the average particle size of the dispersed aqueous phase is at most 200 nm or at most 100 nm or at most 50 nm or at most 20 nm. In some embodiments, the average particle size of the dispersed aqueous phase is at most 1 nm or at most 5 nm or at most 10 nm. The term “particle size” is used to describe the size of micelles of the dispersed phase in a microemulsion. It does not imply that the micelles contain solid particles; the micelles may be partly or wholly liquid.

Each of the components is included in the microemulsion in proportions such that a stable microemulsion forms.
The concentration of water is already discussed.
In some embodiments, the concentration of fatty alcohol or ester in the microemulsion is at least 4 weight percent or at least 5 weight percent or at least 6 weight percent. In some embodiments, the concentration of fatty alcohol or ester in the microemulsion is at most 35 weight percent or at most 25 weight percent or at most 20 weight percent or at most 15 weight percent or at most 10 weight percent.
In some embodiments, the concentration of ethoxylated castor oil in the microemulsion is at least 12 weight percent or at least 15 weight percent or at least 20 weight percent or at least 25 weight percent or at least 30 weight percent. In some embodiments, the concentration of ethoxylated castor oil in the microemulsion is at most 45 weight percent or at most 40 weight percent or at most 30 weight percent or at most 25 weight percent or at most 20 weight percent.
In some embodiments, the concentration of cosolvent in the microemulsion is at least 20 weight percent or at least 25 weight percent or at least 30 weight percent. In some embodiments, the concentration of cosolvent in the microemulsion is at most 65 weight percent or at most 60 weight percent or at most 58 weight percent.
In some embodiments, the concentration of CPE in the microemulsion is at least 5 weight percent or at least 7 weight percent or at least 9 weight percent. In some embodiments, the concentration of CPE in the microemulsion is at most 25 weight percent or at most 20 weight percent or at most 18 weight percent.

Some examples of microemulsions contain:
From 25 to 30 weight percent water;
At least 5 weight percent fatty alcohol or ester or at least 6 weight percent. At most 25 weight percent fatty alcohol or ester or at most 20 weight percent.
At least 15 weight percent ethoxylated castor oil or at least 17 weight percent or at least 18 weight percent. At most 25 weight percent ethoxylated castor oil or at most 22 weight percent.
At least 30 weight percent cosolvent or at least 35 weight percent or at least 37 weight percent. At most 50 weight percent cosolvent or at most 48 weight percent.
At least 6 weight percent CPE or at least 7 weight percent. At most 12 weight percent CPE or at most 10 weight percent.

Some examples of microemulsions contain:
From 20 to 25 weight percent water;
At least 5 weight percent fatty alcohol or ester or at least 6 weight percent. At most 20 weight percent fatty alcohol or ester or at most 18 weight percent.
At least 12 weight percent ethoxylated castor oil or at least 14 weight percent. At most 35 weight percent ethoxylated castor oil or at most 32 weight percent.
At least 25 weight percent cosolvent or at least 27 weight percent. At most 50 weight percent cosolvent or at most 48 weight percent.
At least 6 weight percent CPE or at least 7 weight percent. At most 18 weight percent CPE or at most 15 weight percent.

In some embodiments, the microemulsion can be readily applied to skin and to remain on the skin in liquid form for a time suitable to deliver a drug, such as at least 1 hour or at least 2 hours or at least 3 hours or at least 4 hours. In some embodiments, the microemulsion melts at a temperature below 30° C. or below 25° C. or below 20° C. or below 10° C. or below 0° C. In some embodiments, the microemulsion boils at a temperature above 50° C. or above 75° C. or above 100° C. or above 125° C. or above 150° C.

In some embodiments, a drug is dissolved in the microemulsion in a pharmaceutically-effective concentration. The drug may be dissolved in the microemulsion after it is formed. Alternatively, the drug may be dissolved in a component of the microemulsion before the microemulsion is formed. Suitable drugs and their effective doses are published and known, and suitable drugs are commercially available. Examples of drugs that are commonly administered by skin permeation include lidocaine, scopolamine, nitroglycerine, clonidine, estrogen, testosterone, fentanyl, nicotine, hydrocortisone, ibuprofen, diazepam, tenoxicam, piroxicam, triprolidine, amlodipine, methyl nicotinate, benzocaine, atenolol, tolnaftate, benzoyl peroxide, mupirocin and salicylic acid. In some embodiments, the concentration of the drug in the microemulsion is at least 0.1 weight percent based on the weight of the microemulsion or at least 0.5 weight percent or at least 1 weight percent. In some embodiments, the concentration of the drug in the microemulsion is at most 10 weight percent based on the weight of the microemulsion or at most 8 weight percent or at most 6 weight percent.

The microemulsion may optionally contain other additives useful in topical applications. Examples include thickeners and flow-modifiers, colorants, scents and fillers. In some embodiments, the total concentration of additives excluding fillers is at most 10 weight percent of the total composition or at most 5 weight percent or at most 2 weight percent. Weight percentages listed above for microemulsion components and organic composition components exclude these other additives and particularly exclude fillers.

In some embodiments, the microemulsion compositions of this invention can be stored at 25° C. for up to one month without substantially destabilization, or at least 2 months or at least 3 months or at least 4 months or at least 6 months or at least 1 year.

Microemulsions of the present invention which contain drugs can be used to administer the drug by applying to skin or by infusing into a patch that is adhered to skin. They can be used as sprayable lotions. Gelation agents or viscosity modifiers can be added to the microemulsions to form creams and ointments that can be directly applied to the skin. Use and application is described in patents such as PCT Publication WO 02/09763 and publications such as
Lopes, Overcoming the Cutaneous Barrier with Microemulsions, 6 Pharmaceutics 52-77 (2014);
Kreilgaard, Influence of Microemulsions on Cutaneous Drug Delivery, 54 Advanced Drug Delivery Reviews, Supp. 1, S77-S98 (2002); and
Langley, et al., eds., The Role of Microstructure in Topical Drug Product Development, Springer International Publishing, 2019.

### EXAMPLES

### Measurement Procedures

Micelle size is measured by dynamic light scattering as described in the publication “Surfactant Micelle Characterization using Dynamic Light Scattering” published by Malvern Instruments Ltd (2015), currently available at https://instrumat.ch/wp-content/uploads/2016/08/Surfactant-Micelle-Characterization-AN101104-LRLL.pdf.

### Example 1: Emulsion Studies

A series of solvent mixtures are prepared containing Component A (ethoxylated castor oil), Component B (nonionic surfactant CPE) and one of the Components C1-C9 (cosolvent) in the quantities shown:

| Component | Surfactant | Quantity |
|---|---|---|
| A | TERGITOL ™ ECO36 ethoxylated castor oil | |
| B | Glyceryl oleate (surfactant skin permeation enhancer) | Weight ratio A:B = 2:1 |
| C1 | CARBITOL ™ diethylene glycol monoethyl ether | Weight ratio (A + B):C = 10:6, |
| | | 1:1, 10:15 and 1:2 |
| C2 | Ethoxytriglycerol | Weight ratio (A + B):C = 10:6 |
| C3 | Propylene glycol diacetate (PGDA) | Weight ratio (A + B):C = 10:6 |
| C4 | Tetraethylene glycol | Weight ratio (A + B):C = 10:6 |
| C5 | Poly(ethylene oxide) with molecular weight = 200, | Weight ratio (A + B):C = 10:6 |
| | 300, 400 g/mol | |
| C6 | DOWANOL ™ Hexyl CARBITOL ™ diethylene | Weight ratio (A + B):C = 10:6 |
| | glycol monohexyl ether | |
| C7 | DOWANOL ™ DiEPh diethylene glycol monophenyl | Weight ratio (A + B):C = 10:6 |
| | ether | |
| C8 | DOWANOL ™ PnP 1-propoxy-2-propanol | Weight ratio (A + B):C = 10:6 |
| C9 | 2-ethoxyethanol | Weight ratio (A + B):C = 10:6 |

Each solvent mixture is blended with isopropyl palmitate (fatty ester) in weight ratios of 1:9, 2:8, 3:7, 4:6, 5:5, 6:4, 7:3, 8:2 and 9:1 to form an organic composition.

Each organic composition is blended with water to form a water-in-oil microemulsion in ratios beginning at 2.8 weight percent water and increasing by 2.8 weight percent to 50%. Each microemulsion is blended by shaking for 30 mins using a plate shaker, followed by 90 minutes sonication followed by further shaking for 30 mins. Each microemulsion is rested for about 1 hour and then visually inspected to determine if it is clear and homogeneous (indicating a stable microemulsion) or is clouded or forms strata (indicating that the microemulsion is not stable).

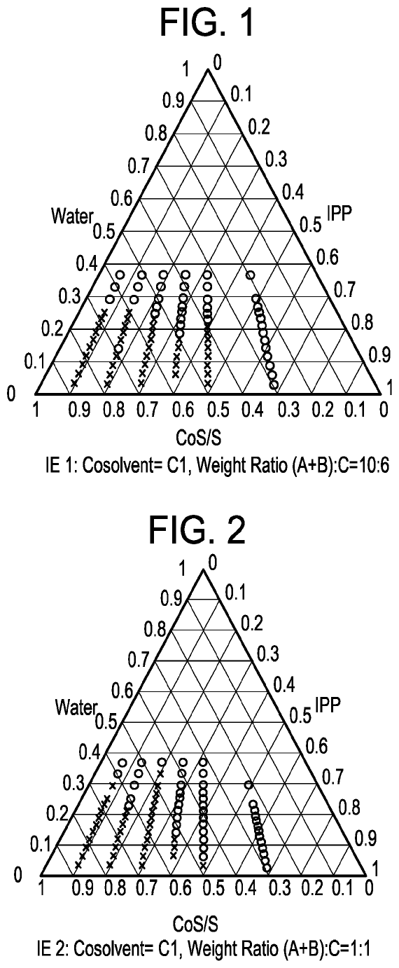

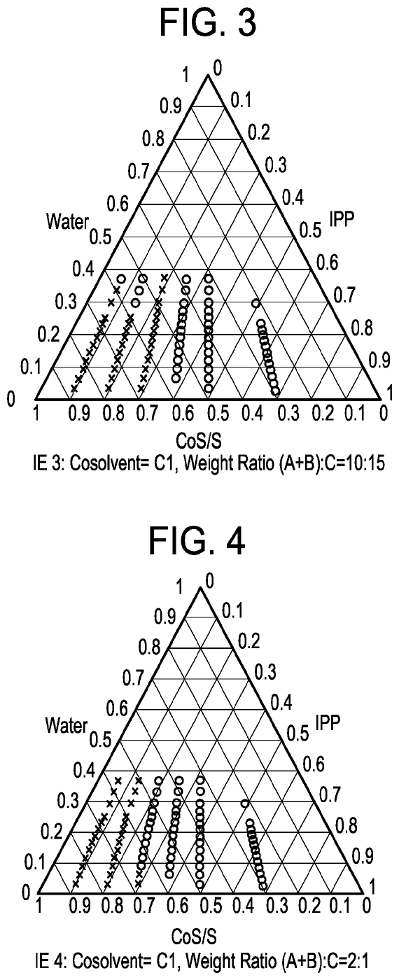

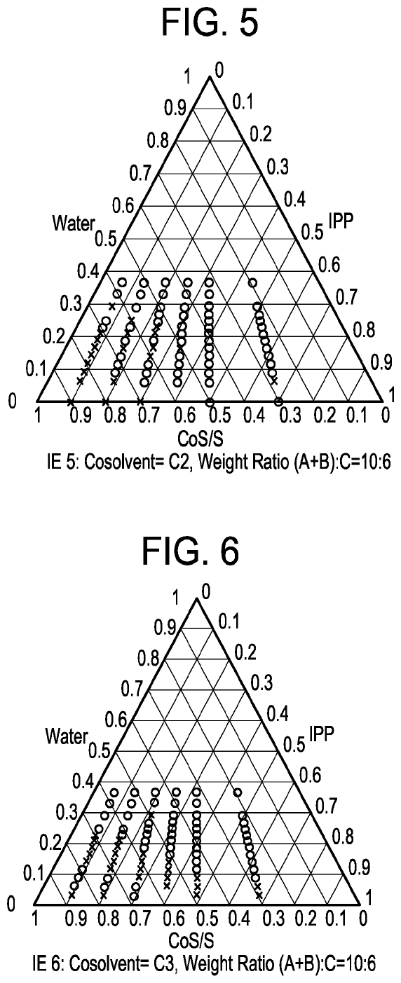

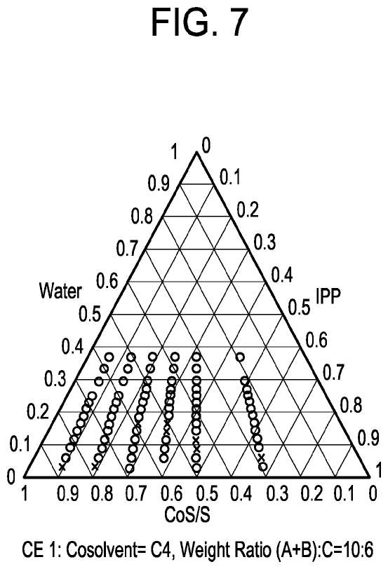

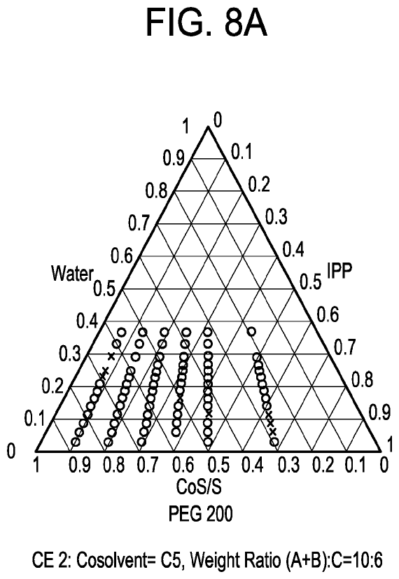

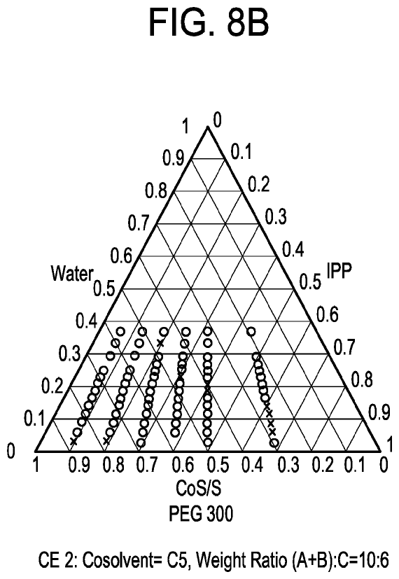

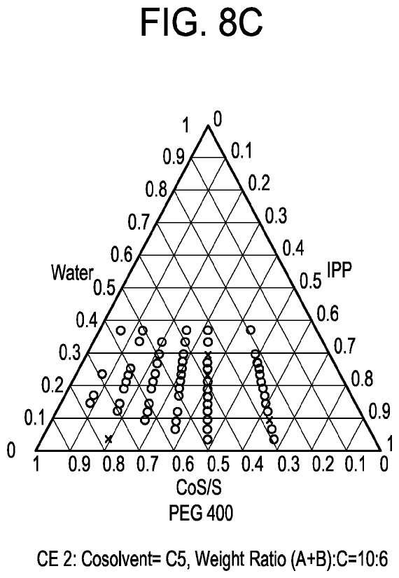

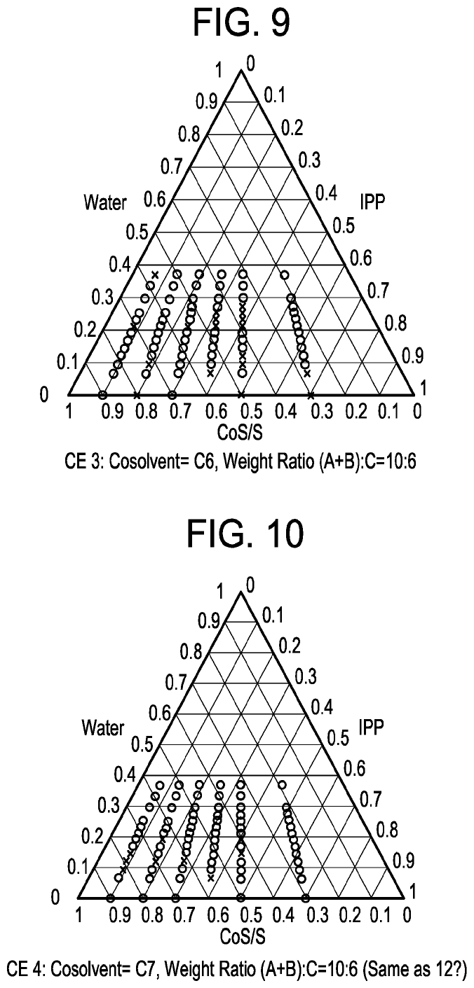

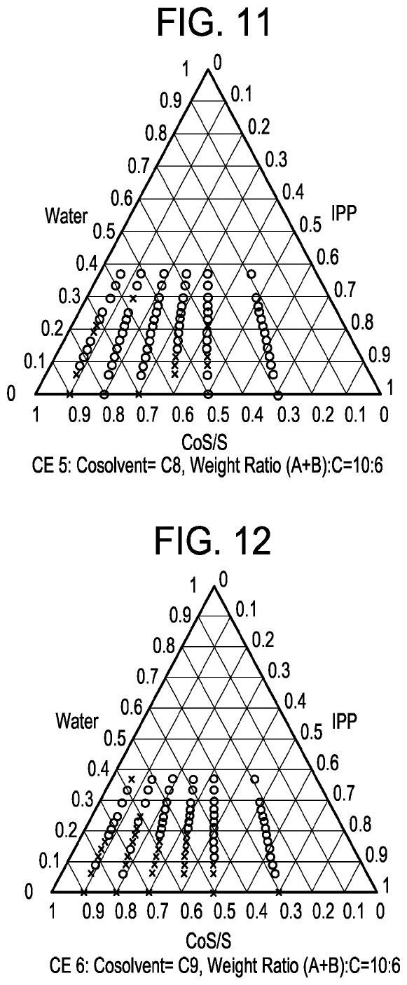

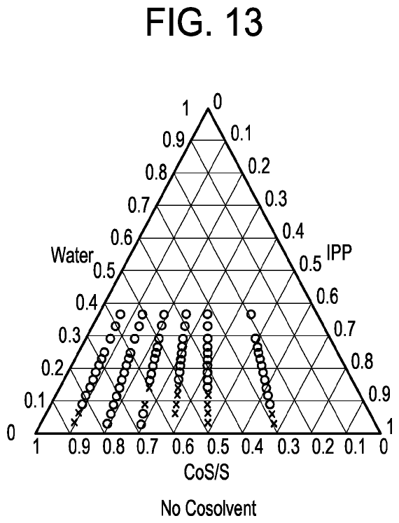

The results are shown in FIGS. 1-13, in which “x” indicates a formulation that forms a stable microemulsion and “o” indicates a formulation that does not form a stable microemulsion, under the conditions of stated in the Examples.

### Example 2: Drug Flux

A group of formulations is made as described in Example 1 that contain 52-54% isopropyl palmitate; 35-37% blend of ethoxylated castor oil (ECO), glyceryl oleate (weight ratio to ECO 1:2) and cosolvent as shown in table 3 below (weight ratio to ECO and glyceryl oleate 10:6); 6-10% water; and 2.5-2.6% lidocaine. A control is also made that contains 2.5 weight percent lidocaine dissolved in isopropyl palmitate.

Drug flux is measured for each formulation using jacketed Franz cells with a receiver volume of 12 mL, which are maintained at 37° C. and area of diffusion of 1.767 cm2. Synthetic polysulfone membrane of 0.45 μm thickness is used as model skin. The receiver is filled with phosphate-buffered saline solution and a 300 μl microemulsion sample is added to the other side of the artificial skin. The concentration of lidocaine in the receiver solution is determined periodically by drawing 250 μl of PBS buffer. The receiver is replenished with the same volume of fresh buffer. The increase in weight of lidocaine in the receiver volume as a function of time yields drug diffusion curves for each microemulsion formulation. All samples are run in triplicates and data was fit between 1-3 h.

| | Formulation | | Flux (μg/h · cm²) |
|---|---|---|---|
| Control | | | 148 ± 24 |
| | Tergitol ™ ECO36 | CARBITOL ™ | 124 ± 8 |
| | Tergitol ™ ECO36 | PEG400 | 155 ± 55 |
| | Tergitol ™ ECO36 | PGDA | 110 ± 68 |
| | Tergitol ™ ECO36 | Tetraethylene glycol | 127 ± 36 |

The results show that the inventive formulations are effective for drug delivery.

We claim:

1. An organic composition that comprises:
a) from 5 to 60 weight percent of fatty alcohol or fatty ester;
b) from 14 to 40 weight percent of ethoxylated castor oil;
c) from 20 to 65 weight percent of a cosolvent selected from the group consisting of lower alkyl diethylene glycol ether, lower alkyl triethylene glycol ether and propylene glycol diacetate; and
d) from 5 to 25 weight percent of a chemical penetration enhancer for skin,
wherein weight percentages are based on the weight of the organic composition, excluding other additives and fillers.

2. The composition of claim 1 wherein the composition comprises no more than 40 weight percent of fatty alcohol or fatty ester.

3. The composition of claim 2 wherein the cosolvent is a lower alkyl diethylene glycol ether or a lower alkyl triethylene glycol ether.

4. The composition of claim 2 wherein the cosolvent is diethylene glycol monoethyl ether or triethylene glycol monoethyl ether.

5. The composition of claim 2 wherein the fatty alcohol or ester is a fatty ester.

6. The composition of claim 1 wherein the concentrations of the components are:
a) From 7 to 15 weight percent fatty alcohol or ester;
b) From 32 to 45 weight percent ethoxylated castor oil;
c) From 24 to 40 weight percent cosolvent; and
d) From 15 to 22 weight percent CPE,
wherein weight percentages are based on the weight of the organic composition, excluding other additives and fillers.

7. The composition of claim 1 wherein the concentrations of the components are:
a) From 7 to 30 weight percent fatty alcohol or ester;
b) From 20 to 35 weight percent ethoxylated castor oil;
c) From 30 to 50 weight percent cosolvent; and
d) From 10 to 20 weight percent CPE,
wherein weight percentages are based on the weight of the organic composition, excluding other additives and fillers.

8. The composition of claim 1 wherein the concentrations of the components are:
a) From 7 to 15 weight percent fatty alcohol or ester;
b) From 18 to 30 weight percent ethoxylated castor oil;
c) From 40 to 60 weight percent cosolvent; and
d) From 8 to 15 weight percent CPE,
wherein weight percentages are based on the weight of the organic composition, excluding other additives and fillers.

9. The composition of claim 1 wherein the concentrations of the components are:
a) From 6 to 20 weight percent fatty alcohol or ester;
b) From 15 to 25 weight percent ethoxylated castor oil;
c) From 45 to 65 weight percent cosolvent; and
d) From 5 to 15 weight percent CPE,
wherein weight percentages are based on the weight of the organic composition, excluding other additives and fillers.

10. The composition of claim 1 which is capable of forming a stable water-in-oil emulsion with a water content from 5 to 30 percent by weight.

11. A water-in-oil microemulsion that contains from 1 to 40 weight percent water and from 60 to 99 weight percent of an organic composition of claim 1.

12. The microemulsion of claim 11 which contains:
a) From 25 to 30 weight percent water;
b) From 5 to 25 weight percent fatty alcohol or ester;
c) From 15 to 25 weight percent ethoxylated castor oil;
d) From 30 weight percent to 50 weight percent cosolvent; and
e) From 6 weight percent to 12 weight percent CPE,
wherein weight percentages exclude other additives and fillers.

13. The microemulsion of claim 11 that contains:
a) From 20 to 25 weight percent water;
b) From 5 to 20 weight percent fatty alcohol or ester;
c) From 12 to 35 weight percent ethoxylated castor oil;
d) From 25 to 50 weight percent cosolvent; and
e) From 6 to 18 weight percent CPE,
wherein weight percentages exclude other additives and fillers.

14. The microemulsion of claim 11 which further comprises a pharmaceutical drug in a pharmaceutically effective concentration.

15. A method to administer a drug by applying to skin a microemulsion of claim 14.
